(19) United States
(12) Patent Application Publication
(10) Pub. No.: US 2016/0338978 A1
(43) Pub. Date: Nov. 24, 2016

(54) Organic Compounds

(71) Applicants: Michael AMBUHL, Rheinfelden (CH); Jutta BEYER, Basel (CH); Begona CARRENO-GOMEZ, Basel (CH); Colleen RUEGGER, Morris Plains, NJ (US); Stephen VALAZZA, Matawan, NJ (US)

(72) Inventors: Michael AMBUHL, Rheinfelden (CH); Jutta BEYER, Basel (CH); Begona CARRENO-GOMEZ, Basel (CH); Colleen RUEGGER, Morris Plains, NJ (US); Stephen VALAZZA, Matawan, NJ (US)

(73) Assignee: Novartis AG, Basel (CH)

(21) Appl. No.: 15/228,722

(22) Filed: Aug. 4, 2016

Related U.S. Application Data
(63) Continuation of application No. 14/255,052, filed on Apr. 17, 2014.
Application No. 14/255,052 is a continuation of application No. 13/553,387, filed on Jul. 19, 2012, now abandoned.
Application No. 13/553,387 is a continuation of application No. 12/442,781, filed on Mar. 25, 2009, now abandoned.

(30) Foreign Application Priority Data
Sep. 26, 2006 (EP) 06121276.7
Sep. 26, 2006 (EP) 06121299.9
Sep. 26, 2006 (EP) 06121301.3
Sep. 26, 2006 (EP) 06121302.1

(51) Int. Cl. A61K 31/137; A61K 9/28; A61K 9/48; A61K 9/20

(52) U.S. Cl. CPC A61K 31/137; A61K 9/2054; A61K 9/2013; A61K 9/2068; A61K 9/2018; A61K 9/2095; A61K 9/2866; A61K 9/282; A61K 9/2063; A61K 9/4808; A61K 9/4833; A61K 9/4866; A61K 9/4858

(57) ABSTRACT

The present invention provides various pharmaceutical compositions comprising an S1P receptor modulator, e.g. an S1P receptor agonist. In one aspect, there is provided a pharmaceutical composition having a coating. In other aspects, rapid disintegrating compositions are provided. In a further aspect, a pharmaceutical composition which is free of sugar alcohols is provided. In another aspect, the invention provides a pharmaceutical composition comprising a coating comprising an S1 Preceptor modulator.

The present invention relates to pharmaceutical compositions comprising a sphingosine-1 phosphate receptor modulator, in particular a sphingosine-1 phosphate receptor agonist.

Sphingosine-1 phosphate (hereinafter referred to as “S1P”) is a natural serum lipid. Presently there are 8 known S1P receptors, namely S1P1 to S1P8. S1P receptor agonists have accelerating lymphocyte homing properties.

S1P receptor agonists are immunomodulating compounds which elicit a lymphopenia resulting from a re-distribution, preferably reversible, of lymphocytes from circulation to secondary lymphatic tissue, evoking a generalized immunosuppression. Naive cells are sequestered, CD4 and CD8 T-cells and B-cells from the blood are stimulated to migrate into lymph nodes (LN) and Peyer's patches (PP), and thus infiltration of cells into transplanted organs is inhibited.

The various known S1P receptor modulators show structural similarities, which result in related problems in providing a suitable formulation. There exists a need for an S1P receptor modulator containing formulation which is well-adapted for oral administration in a solid form, e.g. as a tablet or capsule. In addition, the oral route is often the most convenient route for drug administration, but unfortunately many patients have difficulties in swallowing, e.g. due to an unpleasant taste of the dosage form or there being no water available at the time of ingestion. Thus, there also exists a need for an S1P receptor modulator containing oral formulation which can easily be swallowed, e.g. by children or older patients. Furthermore, there is a need for a way in which to readily produce dosage forms of S1P receptor modulators having a variety of dosage strengths.

The present invention provides various pharmaceutical compositions containing an S1P receptor modulator which address these needs. The compositions provide a convenient means of systemic administration of S1P receptor agonists and other modulators, do not suffer from the disadvantages of liquid formulations for injection or oral use, and have good physicochemical and storage properties. In particular, the compositions of the present invention may show a high level of uniformity in the distribution of the S1P receptor modulator throughout the composition, as well as high stability. The compositions of the invention may be manufactured on high speed automated equipment, and thus do not require hand encapsulation.

In certain aspects, the present invention provides rapid dispersing dosage forms which disintegrate rapidly in the mouth and which do not depend on the use of sweetening or flavoring agents to mask the taste nor do they depend on the presence of a liquid for washing down the dosage form. These dosage forms are capable of disintegrating in the mouth, in particular in saliva. Preferably, the dosage forms have good mouth feel and do not exhibit premature release of the drug in the mouth. Rapid disintegration of the solid pharmaceutical composition may increase the solubility of the active ingredient(s). Particularly in the case of saliva, this may lead to better solubility of the drug than in the small intestine.

The pharmaceutical compositions of the present invention may be produced by standard processes, for instance by conventional mixing, granulating, dissolving or lyophilizing processes. Procedures which may be used are known in the art, e.g. those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed., (Mack Publ., Co., 1970) or later editions.

The compositions of the invention may show good stability characteristics as indicated by standard stability trials, for example having a shelf life stability of up to one, two or three years, and even longer. Preferably, the compositions are stable for at least six months at ambient temperature. Stability characteristics may be determined, e.g. by measuring decomposition products by HPLC analysis after storage for particular times, at particular temperatures, e.g. 20°, 40° or 60° C.

Compositions Comprising a Coating

A pharmaceutical composition can be made easier to swallow by applying a coating to a tablet or pellet cores, or to the surface of a capsule, hence improving compliance by reducing or masking an unpleasant taste.

In one aspect, the present invention provides an oral pharmaceutical composition comprising an S1P receptor modulator, e.g. S1P receptor agonist, wherein the composition comprises a coating comprising:
(a) one or more polymer resins
(b) one or more metal oxides.

Solid compositions may take the form of pellets of differening size, whereby the coating is applied to individual pellets, which may be present in a plurality, for example in a capsule or sachet.

Solid compositions may be formed from powder ingredients, which may be micronised, and may be compressed into compositions of differing hardness.

In one embodiment, the powder constituents of the compressed composition are coated prior to compression.

In another embodiment, the compressed composition is coated after compression.

In another embodiment, the coating is applied both before and after compression.

Liquid oral compositions include capsules containing the liquid composition, where the capsule comprise a coating.

In one embodiment, the coating is applied to the outer surface of the capsule.

In another embodiment, the coating is dispersed within the outer surface of the capsule.

Capsules are not however limited to liquid contents and may comprise solid compositions in the form of powders, pellets or heterogeneous suspensions in addition to homogeneous liquids.

Where the solid composition is in the form of pellets or granules, these may, after application of the coating as described herein, be used as such or to fill capsules, e.g. hard gelatine capsules or other storage means, for example sachets prior to administration.

Pellets and granules may be from 2 to 0.3 mm in diameter, for example, a “normal pellet” has a size of 1 to 0.6 mm and a “bead pellet” has a size of 0.4 to 0.8 mm.

Coating compositions of the present invention are particularly suitable for use on tablet compositions, referred to herein and exemplified as core tablets.

In one embodiment, the coating composition is used to coat a compressed core tablet comprising an S1P modulator, e.g. an S1P agonist.

The core tablet may be any solid formulation for oral administration.

The term “core” comprises, in a wide sense, not only tablets, pellets or granules but also capsules, e.g. soft or hard capsules of gelatine or starch. Such cores may be produced in a conventional manner.

When tablet cores are used they have preferably a hardness of from ca. 10 to 70 N. The tablet core may tensile strength of less than 38 N/cm², for example as low as 22 N/cm².

The hardness of a core tablet comprising an S1P modulator, e.g. an S1P agonist, may be increased by applying a coating as described herein. The coating may therefore provide a means for obtaining tablets having good structural integrity from cores having a tensile strength of less than 38 N/cm² (2.5 kP), i.e. cores that would otherwise have been regarded as too weak for practical use. The cores may have a tensile strength less than 30 N/cm² (2.0 kP), preferably less than 22 N/cm² (1.5 kP).

The cores may be formed by light compression and enable coated components and fragile components, such as capsules, to be used within the compression blend with little or no damage.

The core tablet may comprise an adjuvant and an SW modulator, e.g. an S1P agonist.

The core tablet may comprise conventional tabletting ingredients, including diluents, disintegrants, lubricants, wetting agents, glidants, surfactants, release aids, colourants, gas producers, etc.

The core tablet may be formulated by any known formulation known to the skilled man.

The core tablet may be composed of, but not limited to, fillers such as, polyols, powdered mannitol, for example, or other saccharides or sugars, sugar alcohols etc, e.g. lactose, sucrose, dextrose, mannitol and starch.

The core tablet compositions may also include, or alternatively include, binders such as PVP e.g. cellulose, microcrystalline cellulose, polyethylene glycols, polyvinylpyrrolidone, starch mucilage, acacia, alginic acid, carboxymethylcellulose, hydroxyethylcellulose, hydroxypropylcellulose, dextrin, ethylcellulose, gelatin, glucose, guar gum, hydroxypropylmethylcellulose, magnesium aluminium silicate, kaltodectrin, methylcellulose, polyethylene oxide, povidone, sodium alginate and hydrogenated vegetable oils.

The core tablet compositions may also include, or alternatively include, disintegrants (with or without effervescent agents), e.g. cross-linked sodium carboxymethyl cellulose (crosscarmellose), crosspovidone or sodium starch glycolate.

The core tablet compositions may also include, or alternatively include, lubricants, e.g. magnesium stearate, calcium stearate, sodium stearyl fumarate, colloidal silica or talc.

In one embodiment, the core tablet comprises 1.5 to 2% lubricant, e.g. magnesium stearate or calcium stearate.

The core tablet compositions may also include, or alternatively include, glidants, e.g. silica.

The core tablet compositions may also include, or alternatively include, surfactants, e.g. sodium lauryl sulphate or docusate sodium.

The core tablet compositions may also include, or alternatively include, flavoring agents.

The core tablet compositions may also include, or alternatively include, gas producers, e.g. sodium bicarbonate or citric acid.

The core tablet compositions may also include, or alternatively include, sweeteners.

The core tablet compositions may also include, or alternatively include, pH adjusting agents, e.g. citric acid or fumaric acid.

The core tablet may comprise a release rate controlling additive. For example, the drug may be held within a hydrophobic polymer matrix so that it is gradually leached out of the matrix upon contact with body fluids.

Alternatively, the drug may be held within a hydrophilic matrix which gradually or rapidly dissolves in the presence of body fluid. The tablet core may comprise two or more layers having different release properties. The layers may be hydrophilic, hydrophobic or a mixture of hydrophilic and hydrophobic layers. Adjacent layers in a multilayer tablet core may be separated by an insoluble barrier layer or hydrophilic separation layer. An insoluble barrier layer may be formed of materials used to form the insoluble casing. A hydrophilic separation layer may be formed from a material more soluble than the other layers of the tablet core so that as the separation layer dissolves the release layers of the tablet core are exposed.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, zein etc.

The core tablet may additionally include materials which swell on contact with aqueous liquids, and which may be included in the composition, include polymer materials include from cross-linked sodium carboxymethylcellulose, cross-linked hydroxypropylcellulose, high molecular weighthydroxypropylcellulose, carboxymethylamide, potassium methacrylatedivinylbenzene copolymer, polymethylmethacrylate, cross-linked polyvinylpyrrolidone and high molecular weight polyvinylalcohols.

The core tablet may comprise additional pharmaceutically active ingredients in addition to an S1P modulator, e.g. S1P agonist.

In one embodiment, where the core tablet composition is in unit dosage form, each unit dosage will suitably contain 0.5 to 10 mg of the S1P receptor modulator, e.g. S1P agonist.

Possible manufacturing of the tablet cores comprises blending of all ingredients and further compressing to tablets, and granulation and further compressing of the granules to tablets.

In one embodiment, there is provided a core composition comprising a sugar alcohol. An example of a core tablet comprising an S1P receptor modulator, e.g. S1P agonist, formulation may be found in WO 2004/089341, which describes the formulation of an S1P modulator with a sugar alcohol.

The sugar alcohol may act as a diluent, carrier, filler or bulking agent, and may suitably be mannitol, maltitol, inositol, xylitol or lactitol, preferably a substantially non-hygroscopic sugar alcohol, e.g. mannitol (D-mannitol). A single sugar alcohol may be used, or a mixture of two or more sugar alcohols, e.g a mixture of mannitol and xylitol, e.g. in a ratio of 1:1 to 4:1.

In another embodiment, there is provided a core composition comprising a microcrystalline cellulose and an S1P receptor modulator, e.g. S1P agonist, in the absence of a sugar alcohol.

Preferably, the components of both the core tablet and the coating are micronised.

In one embodiment, the solid formulation may be formulated to have a fast disintegration rate.

Preferably, the active ingredient dose ranges from 0 to 1000 mg.

The coating composition may be powder or liquid based.

The coating composition may have both suitable electrical properties and be fusible at a temperature suitable for use as a coating material in the coating of pharmaceutical tablet cores.

Examples of a polymer resin may include, without limitation, polymethacrylates, for example ammonio methacrylate, cellulose and its derivatives, cellulose ethers and esters and cellulose acetate phthalate.

Preferably, the polymer resin is non-conductive.

The coating composition may comprise polyethylene glycol or a sugar alcohol, e.g. xylitol.

The coating composition may also include, or alternatively include, other possible materials include waxes and oils or alcohols of waxes or oils, poloxamers, alkyl phthalates, for example diethylphthalate, citric acid or esters.

The coating composition may also include, or alternatively include, one or more of acrylic acid, polymers and co-polymers of acrylic acid and their derivatives, for example polymethyl acrylate, polyalkenes and their derivatives, including esters and aryl-esters and their derivatives, polyvinyl alcohols and esters, cellulose and its derivatives, e.g. cellulose ethers and cellulose esters (either cross-linked or uncross-linked) for example ethyl cellulose, and one or more enteric polymers, e.g. cellulose acetate phthalate, hydroxypropyl methyl cellulose phthalate, hydroxypropylcellulose, one or more biodegradable polymers, e.g. one or more of polylactides, polyglycolides, polyhydroxybutyrates, polyhydroxyvalyrate, ethylene vinyl acetate copolymers, and polyanhydrides (homo or hetero polymers), or polyethylene oxide.

The coating composition may also include, or alternatively include, a dispersing agent, e.g. sodium lauryl sulphate, docusate sodium, Tweens (sorbitan fatty acid esters), poloxamers and cetostearylalcohol.

The coating composition may also include, or alternatively include, an anti-friction component to reduce the frictional and/or other forces between the particles of the powder coating material to improve the flowability of the powder, e.g. titanium dioxide, colloidal silicon dioxide, talc or starch or a combination of those.

The coating composition may also include, or alternatively include, a disintegrator, e.g. sodium starch glycolate (cross-linked), sodium carboxymethylcellulose (cross-linked), native starch, cross-linked polyvinyl pyrrolidone (crosprovidone), sodium carbonate, sodium hydrogen carbonate or sodium glycinate.

The coating composition may also include, or alternatively include, colourants, e.g. metal oxides or lakes (e.g. aluminium lakes), iron oxide or dyes.

The coating composition may also include, or alternatively include, taste modifiers, e.g. aspartame, acesulfame k, cyclamates, saccharin, sugars or sugar alcohols.

The coating composition may also include, or alternatively include, flavourings.

In one embodiment, the coating comprises:

(a) a methacrylic acid co-polymer
(b) a cellulose
(c) one or more metal oxides

The present invention also provides a process for producing a coated pharmaceutical composition for oral administration, comprising:

(a) preparing a core tablet comprising an S1P receptor modulator; and
(b) applying a coating as defined above.

In one embodiment, the process comprises:
(a) mixing an S1P receptor agonist or other modulator with a sugar alcohol;
(b) milling and/or granulating the mixture obtained in (a); and
(c) mixing the milled mixture obtained in (b) with a lubricant
(d) optionally, another solvent, a flavor or a preservative, in a propylene glycol and addition of glycerin; and
(e) applying a coating composition of the present invention.

By using this process, a preparation having a good level of content and blend uniformity (e.g. a substantially uniform distribution of the S1P receptor modulator throughout the composition), dissolution time and stability is obtained.

In the case of a tablet core composition comprising the S1P receptor agonist, e.g. 2-amino-2-(2-(4-octylphenyl)ethyl)propane-1,3-diol, hydrochloride, the composition may optionally be micronized, and/or pre-screened, e.g. with a 400 to 500 μm mesh screen, before step (a) in order to remove lumps. The mixing step (a) may suitably comprise blending the S1P receptor agonist and the sugar alcohol, e.g. mannitol in any suitable blender or mixer for e.g. 100 to 400 revolutions.

The process may be carried out by dry mixing the components. In this embodiment the milling step (b) may suitably comprise passing the mixture obtained in (a) through a screen, which preferably has a mesh size of 400 to 500 μm. Process step (a) may comprise the step of mixing the total amount of S1P receptor agonist or other modulator at first with a low amount of sugar alcohol, e.g. from 5 to 25% by weight of the total weight of sugar alcohol, in order to form a pre-mix. Subsequently the remaining amount of sugar alcohol is added to the pre-mix. Step (a) may also comprise the step of adding a binder solution, e.g. methylcellulose and/or xylitol, e.g. an aqueous solution, to the mixture.

The milled mixture obtained in (b) may optionally be blended once more before mixing with the lubricant. The lubricant, e.g. magnesium stearate, is preferably pre-screened, e.g. with a 800 to 900 μm screen, before mixing.

Alternatively, a wet granulation process is employed. In this embodiment, the S1P receptor modulator is preferably first dry-mixed with the desired sugar alcohol, e.g. mannitol, and the obtained sugar alcohol/S1P receptor modulator mixture is then dry-mixed with a binder such as hydroxypropyl cellulose or hydroxypropylmethyt cellulose. Water is then added and the mixture granulated, e.g. using an automated granulator. The granulation is then dried and milled.

If desired, an additional amount of binder may be added in step (c) to the mixture obtained in (b).

The process may comprise a further step of tabletting or encapsulating the mixture obtained in (c), e.g. into a hard gelatin capsule using an automated encapsulation device. The capsules may be coloured or marked so as to impart an individual appearance and to make them instantly recognizable. The use of dyes can serve to enhance the appearance as well as to identify the capsules. Dyes suitable for use in pharmacy typically include carotinoids, iron oxides, and chlorophyll. Preferably, the capsules are marked using a code.

Particularly in the case of coated tablet cores, the coating mixture may be prepared by melt-extrusion of a mixture of polymer, coloring agent and other additives and than further micronization of the produced melt-extrudate is necessary (7 to 10 microns). The coating powders are stable in appropriate packaging and can be used to coat product for at least one year after manufacture.

The coating extending over the tablet core results from the electrostatic deposition of a powder comprising fusible particles.

This technique allows the formation of a thin, continuous film over surface areas of the tablet core. In general, the film will cover from 25 to 100% preferably 50 to 100% of the surface area of the tablet core. The resulting tablet preferably has a tensile strength of at least 50 N/cm², 60 N/cm² and most preferably at least 70 N/cm².

In one embodiment, the following coating process is employed:

First the core is fixed (vacuum) on a wheel, charged, transported through the coating chamber and the opposite charged coating powder is attached to the core surface. Then this powder layered core is transported on the wheel to an IR lamp were the coat melts. Then the core is transferred to the adjacent second wheel and the process is repeated for the bottom part of the tablet core.

Film thickness: 20-50 μm.

Typical coat weights are 3-4% of the core weight eg. 6 mg coat on a 10 mm bi-convex tablet. The max. coat weight for a 12 mm round core is 20 mg. The coat is preferably highly homogenous and preferably has a uniform thickness.

Heating step: This includes heating up the tablets from room temperature, so the temperature at the surface of the tablet peaks at approximately 100° C. and in the tablet core approximately 70° C. for about 20 s. The total thermal exposure is much less that for conventional film coating (60 to 70° C. for 1 to 2 hours).

Preferably, the coating composition is non-conductive and has a melting point below 103° C., e.g. melts within 5 seconds at 130° C.

Preferably, the core is conductive. If it is not conductive, the core preferably contains 3 to 5% of a salt, for example NaCl, KCl, lactilol or citric acid.

In one embodiment, the S1P modulator provides conductive properties to the tablet core.

Therefore, there is provided a process of manufacturing a coated composition comprising an S1P modulator, the process comprising the steps of:

(a) making a composition comprising an S1P modulator, e.g. an S1P agonist
(b) applying an electrostatic coating to the compositing
(c) fixing the coating.

In one particularly preferred process, the S1P modulator represents at least 50% of the conductive component of the core composition, for example at least 60%, typically more than 75%.

The S1P modulator may be the only conductive component in the core composition.

The coating may be also applied by a spaying technique. Conveniently the cores may be treated at room temperature or warmed up to 40° C. e.g. by means of warm air of 40° up to 70° C., before spraying. To avoid sticking of the cores the spray procedure is preferably interrupted at certain time intervals and the cores then warmed up again. It is, however, also possible to proceed without interruption of the spray procedure, e.g. by automatic regulation of the spray amount taking into account the temperature of exhaust air and/or cores.

Various designs, prints, shapes etc may be applied to the coating to provide the final product with a distinctive look.

The spray pressure may vary within wide ranges, in general satisfactory results are obtained with a spray pressure of from about 1 to about 1.5 bar.

Compositions Comprising a Disintegration Agent

Ease of swallowing may also be improved using a fast disintegrating dosage form, e.g. a fast disintegrating tablet.

In another aspect, the invention provides a fast disintegrating solid pharmaceutical composition comprising:
(a) an S1P modulator, e.g. an S1P agonist
(b) an alkaline earth metal silicate
(c) a disintegration agent
   wherein the ratio of the silicate:disintegration agent is from 2:1 to 10:1

Alkaline earth metal silicates include calcium silicate and magnesium silicate.

The disintegrants may additionally comprise effervescent agents.

Examples of disintegrants include, without limitation, crosscarmellose cellulose, crosspovidone and sodium starch glycolate.

The composition may additionally comprise fillers, which may be selected from, for example, gelatin, sugar alcohols, for example, mannitol, sorbitol, dextrose, sucrose, lactose, maltose, sorbitol, maltodextrins, corn syrup solids, or other saccharides or sugars, trehalose, polyvinyl pyrrolidone, polyelectrolyte gel A chondroitin sulfate, cellulose, starch derivatives, pullulan, glycine, docusate Na, PVC, HPC-SL, mannitol & glycerol, gum xanthan/carragean/acacia/guar/tragacanth, mannitol, polysorbate 60, sodium dodecylsulfate, fatty acids, bile salts, sodium methylhydroxybenzoate, sodium propylhydroxybenzoate, polyols, and starch.

The compositions may also include, or alternatively include, lubricants, e.g. magnesium stearate, calcium stearate, sodium stearyl fumarate, colloidal silica or talc.

The composition may additionally comprise additional binders such as PVP, e.g. cellulose, polyethylene glycols, polyvinylpyrrolidone, starch mucilage, acacia, alginic acid, carboxymethylcellulose, hydroxyethylcellulose, hydroxypropylcellulose, hydroxypropylmethylcellulose, dextrin, ethylcellulose, gelatin, glucose, guar gum, hydroxypropylmethylcellulose, magnesium aluminium silicate, kaltodectrin, methylcellulose, polyethylene oxide, povidone, sodium alginate or hydrogenated vegetable oils.

The composition may also include, or alternatively include, surfactants, e.g. sodium lauryl sulphate or docusate sodium.

The composition may also include, or alternatively include, gas producers, e.g. sodium bicarbonate or citric acid.

The composition may additionally, or alternatively, comprise flavoring agents.

The composition may also include, or alternatively include, glidants, e.g. silica.

The composition may additionally, or alternatively, comprise sweeteners.

The composition may additionally, or alternatively, comprise pH adjusting agents, e.g. citric acid or fumaric acid.

In one embodiment, there is provided a composition comprising:

0.1 to 1% S1P modulator, e.g. S1P agonist;
60 to 90% filler, e.g. sugar alcohol;
20 to 45% silicate; and
4 to 10% disintegrant.

Compositions of the present invention may be in the form of, for example, tablets, capsules, caplets, lozenges, pills, mini-tablets, pellets, beads or granules.

Where the solid composition is in the form of pellets or granules, these may be used as such or to fill capsules, e.g. hard gelatine capsules or other storage means, for example sachets prior to administration.

Pellets and granules may be from 2 to 0.3 mm in diameter, for example, a “normal pellet” has a size of 1 to 0.6 mm and a “bead pellet” has a size of 0.4 to 0.8 mm.

The composition may be held within a hydrophilic matrix which gradually or rapidly dissolves in the presence of body fluid.

The composition may additionally include materials which swell on contact with aqueous liquids, and which may be included in the composition, include polymer materials selected from cross-linked sodium carboxymethylcellulose, cross-linked hydroxypropylcellulose, high molecular weight hydroxypropylcellulose, carboxymethylamide, potassium methacrylatedivinylbenzene copolymer, polymethylmethacrylate, cross-linked polyvinylpyrrolidone and high molecular weight polyvinylalcohols.

Preferably, the disintegration time (DT) of the composition is less than 60 seconds upon contact with a fluid, e.g. water or saliva.

Particularly preferably, the DT is about 30 seconds.

Tablet hardness may be adjusted to allow any particular composition have a particular DT. In this respect, compositions of the present invention may have varying hardness.

Accordingly, compositions of the invention may have, for example, a tensile strength of between 30 Nice and 80 N/cm².

Preferably, once disintegrated, the composition is of particle sizes from 1 nm to 10 mm, e.g. 50 nm to 200 nm, which may dissolve or may form a fine suspension.

For a fast disintegration time, the ratio of the silicate, e.g. calcium silicate to disintegrant may be from 2:1 to 10:1, for example 3:1 to 7:1, typically 6:1, 5:1 or 4:1.

In one embodiment, the ratio of calcium silicate to disintegrant is 5:1. For example, the ratio of calcium silicate to crospovidone or croscarmellose may be 5:1.

In one embodiment, there is provided a capsule containing a plurality of pellets having a fast disintegration rate according to the present invention.

The fast disintegration or the improvement in efficiency of disintegration may provide higher solubility of the active substance. Higher solubility of the drug may lead to a higher bioavailability since the risk of precipitation in the body liquid is lower.

The bioavailability of S1P receptor modulators, in particular S1P receptor agonists, may be improved by adding the buccal absorption site to the oral absorption site potentially leading to decrease the first-pass effect. If S1P receptor modulators are buccally absorbed through the sublingual route, the oral mucosa, the esophageal lining and/or the tonsils, bioavailability would be increased as the buccal absorption route circumvents the GI tract (p-gp in the gut) and the first pass liver effect. An increased bioavailability may allow to lower the dose leading to an improved safety profile.

Pharmaceutical dosage forms adapted to supply the medicine to the oral cavity for buccal, sublingual or gingival absorption may be used with and without the presence of enhancer agents such as, but not limited to, those described in the Examples.

Examples of these dosage forms include but are not limited to: buccal spray, effervescent tablets, granules, orally disintegrating tablets, thin films or wafers and mucoadhesive discs or patches.

Preferably, the active ingredient dose ranges from 0 to 1000 mg, for example 0 to 500 mg.

Compositions Comprising a Freeze Dried Dosage Form

In another aspect, the present invention provides a rapid disintegrating pharmaceutical composition comprising a freeze dried dosage form of an S1P modulator, e.g. an S1P agonist.

In one embodiment, the compositions comprise a freeze-dried dosage form comprising one or more S1P modulator, e.g. S1P agonist, particles which may be uncoated or coated with a polymer or lipid material which exhibit minimal release of the drug in the mouth.

This may be achieved, for example, by using coarse coated drug particles and controlling the viscosity of the suspension by reducing the temperature during the holding time in suspension to minimize sedimentation of the particles without altering the physical properties of the dried units.

The resulting dosage form exhibits delayed release of the drug for a time at least sufficient to mask the taste in the mouth before swallowing, and typically for a longer period of time to provide controlled or sustained release of the drug after swallowing.

The carrier material, which forms a network or matrix containing the S1P modulator, e.g. S1P agonist, after freeze drying, may be any water-soluble or water-dispersible material that is pharmaceutically acceptable, inert to the pharmaceutically active substance and which is capable of forming a rapidly disintegrating network, i.e. disintegrates within, for example 10 seconds or less in the mouth.

An effect of the freeze dried dosage form is that the dosage form is highly dispersed and as a consequence is able to disintegrate rapidly. As a result the compositions may form fine suspensions or solutions on contact with saliva in the mouth.

A preferred carrier material is gelatin, usually pharmaceutical grade gelatin. Other substances may be used as the carrier material include, for example, hydrolyzed dextrose, dextran, dextrin, maltodextrin, alginates, hydroxyethyl cellulose, sodium carboxymethyl cellulose, microcrystalline cellulose, corn-syrup solids, pectin, carrageenan, agar, chitosan, locust bean gum, xanthan gum, guar gum, acacia gum, tragacanth, conjac flower, rice flower, wheat gluten, sodium starch glycolate, soy fiber protein, potato protein, papain, horseradish peroxidase, glycine and mannitol.

The composition of may also comprise additional excipients, which may be, for example a cellulose or a sugar alcohol.

Additional excipients, where not in use as a carrier material may nevertheless be used and may be selected from for example, sugar alcohols, for example, mannitol, sorbitol, dextrose, sucrose, lactose, maltose, sorbitol, maltodextrins, corn syrup solids, trehalose, polyvinyl pyrrolidone, polyelectrolyte gel A chondroitin sulfate, cellulose, starch derivatives, pullulan, glycine, docusate Na, PVC, HPC-SL, mannitol & glycerol, gum xanthan/carragean/acacia/guar/tragacanth, mannitol, polysorbate 60, sodium dodecylsulfate, fatty acids, bile salts, sodium methylhydroxybenzoate, sodium propylhydroxybenzoate, polyols, and starch.

The composition may be held within a hydrophilic matrix which gradually or rapidly dissolves in the presence of body fluid.

The composition may additionally include materials which swell on contact with aqueous liquids, and which may be included in the composition, include polymer materials selected from cross-linked sodium carboxymethylcellulose, cross-linked hydroxypropylcellulose, high molecular weighthydroxypropylcellulose, carboxymethylamide, potassium methacrylatedivinylbenzene copolymer, polymethylmethacrylate, cross-linked polyvinylpyrrolidone and high molecular weight polyvinylalcohols.

In one embodiment, the composition comprises gelatin and a polysaccharide, e.g. Pullulan or a sugar alcohol and a freeze dried dosage form of an S1P receptor agonist or other modulator.

In a particular embodiment, the sugar alcohol acts as a structure forming agent.

In another embodiment, the gelatin and the sugar alcohol are present in a ratio of from 3:1 to 1:3, for example 2:1 to 1:2, typically 1:1.

In a further embodiment, the gelatin is present in an amount of 2 to 10%, for example 2 to 4% and the sugar alcohol is present in an amount of 0.1 to 15%, for example 0.5 to 8%.

The composition may also include, or alternatively include, binders such as PVP, e.g. cellulose, polyethylene glycols, polyvinylpyrrolidone, starch mucilage, acacia, alginic acid, carboxymethylcellulose, hydroxyethylcellulose, hydroxypropylcellulose, hydroxypropylmethylcellutose, dextrin, ethylcellulose, gelatin, glucose, guar gum, hydroxypropylmethylceliulose, magnesium aluminium silicate, kaltodectrin, methylcellulose, polyethylene oxide, povidone, sodium alginate or hydrogenated vegetable oils.

The composition may also include, or alternatively include, disintegrants (with or without effervescent agents), e.g. cross-linked sodium carboxymethyl cellulose (crosscarmellose), crosspovidone or sodium starch glycolate.

The compositions may also include, or alternatively include, lubricants e.g. stearic acid, magnesium stearate, calcium stearate, zinc stearate, glyceryl palmitostearate, sodium stearyl fumarate, canola oil, hydrogenated vegetable oil such as hydrogenated castor oil (e.g. Cutina® or Lubriwax® 101), mineral oil, sodium lauryl sulfate, magnesium oxide, colloidal silicon dioxide, polyethylene glycol, polyvinyl alcohol, sodium benzoate, talc, poloxamer, or a mixture of any of the above.

The composition may also include, or alternatively include, surfactants, e.g. sodium lauryl sulphate, docusate sodium.

The composition may also include, or alternatively include, glidants, e.g. silica.

The composition may also include, or alternatively include, flavoring agents.

The composition may also include, or alternatively include, gas producers, e.g. sodium bicarbonate or citric acid.

The composition may also include, or alternatively include, sweeteners.

The composition may also include, or alternatively include, pH adjusting agents, e.g. citric acid or fumaric acid.

The composition may also include, or alternatively include, viscosity enhancers.

Compositions of the present invention may be in the form of, for example, tablets, capsules, caplets, lozenges, pills, mini-tablets, pellets, beads or granules.

Where the solid composition is in the form of pellets or granules, these may, after application of the coating as described hereinafter, be used as such or to fill capsules, e.g. hard gelatine capsules or other storage means, for example sachets prior to administration.

Pellets and granules may be from 2 mm to 0.3 mm in diameter, for example, a “normal pellet” has a size of 1 to 0.6 mm and a “bead pellet” has a size of 0.4 to 0.8 mm.

In one embodiment, there is provided a capsule containing a plurality of pellets having a rapid disintegration rate according to the present invention.

Rapid disintegration, or more efficient may provide higher solubility of the active substance. Higher solubility of the drug may lead to a higher bioavailability since the risk of precipitation in the body liquid is lower.

The term “rapid disintegration” as used herein means that the solid dosage form will disintegrate in water at 37° C. in 60 seconds or less. The forms usually disintegrate in about 5 to 20 seconds, more usually 5 to 10 seconds or less, when tested by the following procedure which is analogous to the Disintegration Test for Tablets, B. P. 1973 which is described in British patent number 1548022.

The bioavailability of S1P receptor modulators, in particular S1P receptor agonists, may be improved by adding the buccal absorption site to the oral absorption site potentially leading to decrease the first-pass effect. If S1P receptor modulators are buccally absorbed through the sublingual route, the oral mucosa, the esophageal lining and/or the tonsils, bioavailability would be increased as the buccal absorption route circumvents the GI tract (p-gp in the gut) and the first pass liver effect. An increased bioavailability may allow to lower the dose leading to an improved safety profile.

Pharmaceutical dosage forms adapted to supply the medicine to the oral cavity for buccal, sublingual or gingival absorption will be used with and without the presence of enhancer agents such as, but not limited to, those described in the Examples.

Examples of these dosage forms include but are not limited to: buccal spray, effervescent tablets, granules, orally disintegrating tablets, thin films or wafers and mucoadhesive discs or patches.

Preferably, the active Ingredient dose ranges from 0 to 1000 mg, for example 0 to 500 mg.

The dosage forms can be manufactured by known means, resulting in suspensions and the like. Liquid suspensions are then poured into discrete units, for example contained within the pockets of a suitable mold. Alternatively, the suspension may be in the form of solid units, for example frozen units or gelled units where the carrier material readily forms a gel. Typically each unit will contain up to 250 mg of the drug, for example 10 to 100 mg. Unit dosage forms of the drug in rapidly disintegrating form are encompassed by the present invention.

The suspension of the particles in the carrier material is preferably formed into discrete units by introduction into a mold which preferably comprises a plurality of depressions, each of the depressions being of the desired shape and size for the oral dosage form product. The mold preferably comprises a plurality of depressions formed in sheet of a filmic material which may be similar to the material employed conventionally in the blister packaging of pharmaceuticals.

Alternative methods of forming discrete frozen or gelled units of the suspension include solidifying the mixtures in dropwise fashion. For example, the suspension may be passed through one or more holes to form drops, spheres or a spray of small particles which can be solidified by passage through a cold gas or liquid, for example liquid nitrogen. Alternatively, the drops, spheres or spray may be solidified by contact with a chilled liquid which is immiscible with the solution or suspension and which has a density such that the drops either fall through the immiscible liquid as they solidify or float on the surface of the immiscible liquid.

Removal of the continuous phase from the discrete units of the suspension comprising the pharmaceutically active substance is carried out by techniques well known to those skilled in the art. For example, when the discrete units are in a liquid form, they will generally be frozen or gelled prior to drying. The suspension contained within the pockets of a suitable mold is frozen, for example by passing a gaseous cooling medium such as liquid nitrogen over the mold or by inserting the mold into a nitrogen spray freezing chamber. Alternatively, the mold may be cooled by passing the mold over a cold surface. Once the dosage forms have been frozen, the mold may be stored in a cold store prior to drying.

Frozen discrete units may be dried by freeze drying according to techniques which are well known in the art. The continuous phase, for example water, is sublimed in a freeze drying process under a reduced pressure which transforms the solid phase solvent (ice) directly into a vapor. The freeze drying process will generally be carried out in a freeze drying chamber typically operating under a vacuum of 0.1 to 1.0 mBar for a period of time of from 180 to 500 minutes.

The present invention also provides a process for producing a pharmaceutical composition, comprising:
(a) mixing a freeze dried dosage form of an S1P receptor agonist or other modulator with a structure forming agent;
(b) producing an aqueous suspension, wherein the aqueous suspension contains less than 50% solid; and
(c) optionally further conducting a lyophillisation step.

In one embodiment, the suspension is cooled to 10 to 20° C., for example 15° C., prior to the a lyophillisation step.

Compositions in which Sugar Alcohol is Absent

In a further aspect, the invention provides a solid pharmaceutical composition suitable for oral administration, comprising:

(a) a S1P receptor modulator, e.g. an S1P agonist; and
(b) a microcrystalline cellulose
in the absence of a sugar alcohol.

The composition may further comprise a lubricant.

Suitable lubricants include stearic acid, magnesium stearate, calcium stearate, zinc stearate, glyceryl palmitostearate, sodium stearyl fumarate, canola oil, hydrogenated vegetable oil such as hydrogenated castor oil (e.g. Cutina® or Lubriwax® 101), mineral oil, sodium lauryl sulfate, magnesium oxide, colloidal silicon dioxide, polyethylene glycol, polyvinyl alcohol, sodium benzoate, talc, poloxamer, or a mixture of any of the above.

Preferably the lubricant comprises magnesium stearate or a hydrogenated vegetable oil.

The composition preferably contains 0.01 to 5% by weight of the lubricant, more preferably 1 to 3% by weight, e.g. about 2% by weight, based on the total weight of the composition.

The composition may comprise one or more further excipients such as carriers, binders or diluents.

The composition may comprise an additional binder for example, methylcellulose, hydroxypropylcellulose, hydroxypropylmethylcellulose, dicalcium phosphate, PVP, e.g. cellulose, polyethylene glycols, polyvinylpyrrolidone, starch mucilage, acacia, alginic acid, carboxymethylcellulose, hydroxyethylcellulose, dextrin, ethylcellulose, gelatin, guar gum, hydroxypropylmethylcellulose, magnesium aluminium silicate, kaltodectrin, methylcellulose, polyethylene oxide, povidone, sodium alginate or hydrogenated vegetable oils.

The composition may also include, or alternatively include, glidants, e.g. silica.

The composition may be in form of a powder, granule or pellets or in unit dosage form, for example as a tablet or capsule. The compositions are well-adapted for encapsulation into an orally administrable capsule shell, particularly a hard gelatin shell. Alternatively the compositions may be compacted into tablets.

Tablets may be coated, for instance with talc or a polysaccharide (e.g. cellulose) or hydroxypropylmethylcellulose coating.

The composition may also additionally comprise disintegrants. Examples of disintegrants are, for example, crosscarmellose cellulose, crosspovidone and sodium starch glycolate.

The composition may also include, or alternatively include, surfactants, e.g. sodium lauryl sulphate or docusate sodium.

The composition may also include, or alternatively include, gas producers, e.g. sodium bicarbonate or citric acid.

The composition may comprise a release rate controlling additive. For example, the drug may be held within a hydrophobic polymer matrix so that it is gradually leached out of the matrix upon contact with body fluids.

Alternatively, the drug may be held within a hydrophilic matrix which gradually or rapidly dissolves in the presence of body fluid. The tablet core may comprise two or more layers having different release properties. The layers may be hydrophilic, hydrophobic or a mixture of hydrophilic and hydrophobic layers. Adjacent layers in a multilayer tablet core may be separated by an insoluble barrier layer or hydrophilic separation layer. An insoluble barrier layer may be formed of materials used to form the insoluble casing. A hydrophilic separation layer may be formed from a material more soluble than the other layers of the tablet core so that as the separation layer dissolves the release layers of the tablet core are exposed.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, zein etc.

The composition may additionally include materials which swell on contact with aqueous liquids, and which may be included in the composition, include polymer materials selected from cross-linked sodium carboxymethylcellulose, cross-linked hydroxypropylcellulose, high molecular weighthydroxypropylcellulose, carboxymethylamide, potassium methacrylatedivinylbenzene copolymer, polymethylmethacrylate, cross-linked polyvinylpyrrolidone and high molecular weight polyvinylalcohols.

In one embodiment, the composition includes a silicon dioxide.

The microcrystalline cellulose may act as a diluent, carrier, filler or bulking agent, and may suitably be Avicel®. The size of the particles of the microcrystalline cellulose may vary.

The use of microcrystalline cellulose composition may assist in promoting uniform distribution of the S1P receptor modulator throughout the microcrystalline cellulose in the composition. A higher surface area may be achieved by providing a microcrystalline cellulose preparation consisting of particles having a smaller mean size and/or a rougher surface on each particle.

The use of micronized microcrystalline cellulose, e.g. with a mean particle size of 30 μm or less, has also been found to improve compressibility and hardness of tablets formed from the composition.

The composition preferably contains 75 to 99.99% by weight of the microcrystalline cellulose, e.g. 85 to 99.9%, e.g. 90 to 99.5% by weight, based on the total weight of the composition.

Typically sugar alcohols include lactose, sucrose, dextrose, mannitol or sorbitol.

Compositions of the present invention may be in the form of, for example, tablets, capsules, caplets, lozenges, pills, mini-tablets, pellets, beads or granules.

Where the solid composition is in the form of pellets or granules, these may, after application of the coating as described hereinafter, be used as such or to fill capsules, e.g. hard gelatine capsules or other storage means, for example sachets prior to administration.

Pellets and granules may be from 2 to 0.3 mm in diameter, for example, a “normal pellet” has a size of 1 to 0.6 mm and a “bead pellet” has a size of 0.4 to 0.8 mm

The present invention also provides a process for producing a pharmaceutical composition, comprising:

(a) mixing an S1P receptor agonist or other modulator with a microcrystalline cellulose, e.g. Avicel®;
(b) milling and/or granulating the mixture obtained in (a); and
(c) optionally mixing the milled mixture obtained in (b) with a lubricant.

By using this process, a preparation having a good level of content and blend uniformity (i.e. a substantially uniform distribution of the S1P receptor modulator throughout the composition), dissolution time and stability is obtained.

The S1P receptor modulator, e.g. 2-amino-2-[2-(4-octylphenyl)ethyl]propane-1,3-diol or other S1P receptor agonist, hydrochloride, may optionally be micronized, and/or pre-screened, e.g. with a 400 to 500 μm mesh screen, before step (a) in order to remove lumps. The mixing step (a) may suitably comprise blending the S1P receptor agonist and the microcrystalline cellulose, e.g. Avicel®, in any suitable blender or mixer for e.g. 100 to 400 revolutions.

The process may be carried out by dry mixing the components. In this case, the milling step (b) may suitably comprise passing the mixture obtained in (a) through a screen, which preferably has a mesh size of 400 to 500 μm. Process step (a) may comprise the step of mixing the total amount of S1P receptor agonist at first with a low amount of microcrystalline cellulose, e.g. Avicel®, e.g. from 5 to 25% by weight of the total weight of microcrystalline cellulose, e.g. Avicel®, in order to form a pre-mix. Subsequently the remaining amount of microcrystalline cellulose, e.g. Avicel®, is added to the pre-mix. Step (a) may also comprise the step of adding a binder solution, e.g. methylcellulose and/or xylitol, e.g. an aqueous solution, to the mixture.

The milled mixture obtained in (b) may optionally be blended once more before mixing with the lubricant. The lubricant, e.g. magnesium stearate, is preferably pre-screened, e.g. with a 800 to 900 μm screen, before mixing.

Alternatively, a wet granulation process is employed. In this embodiment, the S1P receptor modulator is preferably first dry-mixed with the desired microcrystalline cellulose, e.g. Avicel®, and the obtained microcrystalline cellulose, e.g. Avicel®/S1P receptor modulator mixture is then dry-mixed with a binder such as hydroxypropyl cellulose or hydroxypropylmethyl cellulose. Water is then added and the mixture granulated, e.g. using an automated granulator. The granulation is then dried and milled.

If desirable, an additional amount of binder may be added in step (c) to the mixture obtained in (b).

The process may comprise a further step of tabletting or encapsulating the mixture obtained in (c), e.g. into a hard gelatin capsule using an automated encapsulation device. The capsules may be coloured or marked so as to impart an individual appearance and to make them instantly recognizable. The use of dyes can serve to enhance the appearance as well as to identify the capsules. Dyes suitable for use in pharmacy typically include carotinoids, iron oxides, and chlorophyll. Preferably, the capsules are marked using a code.

Compositions Comprising a Coating Comprising an S1P Receptor Agonist

By applying a coating comprising an S1P receptor modulator to a pharmaceutical composition, different dosage strengths or combination products may be formulated.

Accordingly, in a further aspect the present invention provides a pharmaceutical composition which comprises a coating comprising an S1P receptor modulator, e.g. an S1P receptor agonist.

The pharmaceutical composition generally comprises a core coated with a coating comprising an S1P receptor modulator, e.g. an S1P receptor agonist.

The core may be any solid formulation for oral administration.

The term “core” comprises, in a wide sense, not only tablets, pellets or granules but also capsules, e.g. soft or hard capsules of gelatine or starch. In particular, the core may be a granule, pellet, tablet or minitablet. Such cores may be produced in a conventional manner.

In embodiments, the core also contains an S1P receptor modulator, e.g. S1P receptor agonist. In other embodiments, an S1P receptor agonist is absent from the core.

Solid compositions may take the form of pellets of differening size, whereby the coating is applied to individual pellets, which may be present in a plurality, for example in a capsule or sachet.

Solid compositions may be formed from powder ingredients, which may be micronised, and may be compressed into compositions of differing hardness.

In one embodiment, the powder constituents of the compressed composition are coated prior to compression.

In another embodiment, the compressed composition is coated after compression.

In another embodiment, the coating is applied both before and after compression.

Liquid oral compositions include capsules containing the liquid composition, where the capsule comprise a coating.

In one embodiment, the coating is applied to the outer surface of the capsule.

In another embodiment, the coating is dispersed within the outer surface of the capsule.

Capsules are not however limited to liquid contents and may comprise solid compositions in the form of powders, pellets or heterogeneous suspensions in addition to homogeneous liquids.

Where the solid composition Is in the form of pellets or granules, these may, after application of the coating as described herein, be used as such or to fill capsules, e.g. hard gelatine capsules or other storage means, for example sachets prior to administration.

Pellets and granules may be from 2 to 0.3 mm in diameter, for example, a “normal pellet” has a size of 1 to 0.6 mm and a “bead pellet” has a size of 0.4 to 0.8 mm.

Coating compositions of the present invention are particularly suitable for use on tablet compositions, referred to herein and exemplified as core tablets.

In one embodiment, the coating composition is used to coat a compressed core tablet comprising an S1P modulator, e.g. an S1P agonist.

When tablet cores are used they have preferably a hardness of from ca. 10 to 70 N. The tablet core may tensile strength of less than 38 N/cm², for example as low as 22 N/cm².

The cores may be formed by light compression and enable coated components and fragile components, such as capsules, to be used within the compression blend with little or no damage.

The core may comprise an adjuvant and an S1P modulator, e.g. an S1P agonist.

The core may comprise conventional tabletting ingredients, including diluents, disintegrants, lubricants, wetting agents, glidants, surfactants, release aids, colourants, gas producers, etc.

The core may be formulated by any known formulation known to the skilled man.

The core may be composed of, but not limited to, fillers such as, polyols, powdered mannitol, for example, or other saccharides or sugars, sugar alcohols etc, e.g. lactose, sucrose, dextrose, mannitol and starch.

The core compositions may also include, or alternatively include, binders such as PVP e.g. cellulose, microcrystalline cellulose, polyethylene glycols, polyvinylpyrrolidone, starch mucilage, acacia, alginic acid, carboxymethylcellulose, hydroxyethylcellulose, hydroxypropylcellulose, dextrin, ethylcellulose, gelatin, glucose, guar gum, hydroxypropylmethylcellulose, magnesium aluminium silicate, kaltodectrin, methylcellulose, polyethylene oxide, povidone, sodium alginate and hydrogenated vegetable oils.

The core compositions may also include, or alternatively include, disintegrants (with or without effervescent agents), e.g. cross-linked sodium carboxymethyl cellulose (crosscarmellose), crosspovidone or sodium starch glycolate.

The core compositions may also include, or alternatively include, lubricants, e.g. magnesium stearate, calcium stearate, sodium stearyl fumarate, colloidal silica or talc.

In one embodiment, the core comprises 1.5 to 2% lubricant, e.g. magnesium stearate or calcium stearate.

The core compositions may also include, or alternatively include, glidants, e.g. silica.

The core compositions may also include, or alternatively include, surfactants, e.g. sodium lauryl sulphate or docusate sodium.

The core compositions may also include, or alternatively include, flavoring agents.

The core compositions may also include, or alternatively include, gas producers, e.g. sodium bicarbonate or citric acid.

The core compositions may also include, or alternatively include, sweeteners.

The core compositions may also include, or alternatively include, pH adjusting agents, e.g. citric acid or fumaric acid.

The core may comprise a release rate controlling additive. For example, the drug may be held within a hydrophobic polymer matrix so that it is gradually leached out of the matrix upon contact with body fluids.

Alternatively, the drug may be held within a hydrophilic matrix which gradually or rapidly dissolves in the presence of body fluid. The core may comprise two or more layers having different release properties. The layers may be hydrophilic, hydrophobic or a mixture of hydrophilic and hydrophobic layers. Adjacent layers in a multilayer core may be separated by an insoluble barrier layer or hydrophilic separation layer. An insoluble barrier layer may be formed of materials used to form the insoluble casing. A hydrophilic separation layer may be formed from a material more soluble than the other layers of the tablet core so that as the separation layer dissolves the release layers of the tablet core are exposed.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, zein etc.

The core may additionally include materials which swell on contact with aqueous liquids, and which may be included in the composition, include polymer materials include from cross-linked sodium carboxymethylcellulose, cross-linked hydroxypropylcellulose, high molecular weighthydroxypropylcellulose, carboxymethylamide, potassium methacrylatedivinylbenzene copolymer, polymethylmethacrylate, cross-linked polyvinylpyrrolidone and high molecular weight polyvinylalcohols.

The core may comprise additional pharmaceutically active ingredients in addition to an S1P modulator, e.g. S1P agonist.

In one embodiment, where the core composition is in unit dosage form, each unit dosage will suitably contain 0.5 to 10 mg of the S1P receptor modulator, e.g. S1P agonist.

Possible manufacturing of tablet cores comprises blending of all ingredients and further compressing to tablets, and granulation and further compressing of the granules to tablets.

In one embodiment, there is provided a core composition comprising a sugar alcohol. An example of a core tablet comprising an S1P receptor modulator, e.g. S1P agonist, formulation may be found in WO 2004/089341, which describes the formulation of an S1P modulator with a sugar alcohol.

The sugar alcohol may act as a diluent, carrier, filler or bulking agent, and may suitably be mannitol, maltitol, inositol, xylitol or lactitol, preferably a substantially non-hygroscopic sugar alcohol, e.g. mannitol (D-mannitol). A single sugar alcohol may be used, or a mixture of two or more sugar alcohols, e.g a mixture of mannitol and xylitol, e.g. in a ratio of 1:1 to 4:1.

In another embodiment, there is provided a core composition comprising a microcrystalline cellulose and an S1P receptor modulator, e.g. S1P agonist, in the absence of a sugar alcohol.

Preferably, the components of both the core and the coating are micronised.

In one embodiment, the solid formulation may be formulated to have a fast disintegration rate.

Preferably, the active ingredient dose ranges from 0 to 1000 mg.

The coating composition may be powder or liquid based.

The coating composition may comprise a polymer resin.

Examples of a polymer resin may include, without limitation, polymethacrylates, for example ammonio methacrylate, cellulose and its derivatives, cellulose ethers and esters and cellulose acetate phthalate.

The coating composition may comprise polyethylene glycol or a sugar alcohol, e.g. xylitol.

The coating composition may also include, or alternatively include, other possible materials include waxes and oils or alcohols of waxes or oils, poloxamers, alkyl phthalates, for example diethylphthalate, citric acid or esters.

The coating composition may also include, or alternatively include, one or more of acrylic acid, polymers and co-polymers of acrylic acid and their derivatives, for example polymethyl acrylate, polyalkenes and their derivatives, including esters and aryl-esters and their derivatives, polyvinyl alcohols and esters, cellulose and its derivatives, e.g. cellulose ethers and cellulose esters (either cross-linked or uncross-linked) for example ethyl cellulose, and one or more enteric polymers, e.g. cellulose acetate phthalate, hydroxypropyl methyl cellulose phthalate, hydroxypropylcellulose, one or more biodegradable polymers, e.g. one or more of polylactides, polyglycolides, polyhydroxybutyrates, polyhydroxyvalyrate, ethylene vinyl acetate copolymers, and polyanhydrides (homo or hetero polymers), or polyethylene oxide.

The coating composition may also include, or alternatively include, a dispersing agent, e.g. sodium lauryl sulphate, docusate sodium, Tweens (sorbitan fatty acid esters), poloxamers and cetostearylalcohol.

The coating composition may also include, or alternatively include, an anti-friction component to reduce the frictional and/or other forces between the particles of the powder coating material to improve the flowability of the powder, e.g. titanium dioxide, colloidal silicon dioxide, talc or starch or a combination of those.

The coating composition may also include, or alternatively include, a disintegrator, e.g. sodium starch glycolate (cross-linked), sodium carboxymethylcellulose (cross-linked), native starch, cross-linked polyvinyl pyrrolidone (crosprovidone), sodium carbonate, sodium hydrogen carbonate or sodium glycinate.

The coating composition may also include, or alternatively include, colourants, e.g. metal oxides or lakes (e.g. aluminium lakes), iron oxide or dyes.

The coating composition may also include, or alternatively include, taste modifiers, e.g. aspartame, acesulfame k, cyclamates, saccharin, sugars or sugar alcohols.

The coating composition may also include, or alternatively include, flavourings.

The composition may comprise one or more further coatings. The composition may be separated from the drug-containing coating by a protection coating. Alternatively or additionally, the drug-containing coating may be coated by an overcoat. The or each further coating may comprise a polymer material, for example hydroxypropylmethylcellulose or hydroxypropylcellulose. Such coatings may be produced and applied to the composition using techniques known in the art.

The present invention also provides a process for producing a coated pharmaceutical composition, comprising:

(a) preparing a core composition; and
(b) coating the core with a coating comprising an S1P receptor modulator.

The core composition may be prepared using any of the techniques described herein.

The coating may be applied to the core using techniques well known in the art, for example by a fluidized bed process.

S1P Modulators

Each of the various compositions described herein comprises an S1P modulator. In embodiments of each of the compositions described herein, the S1P modulator is an S1P agonist.

S1P receptor agonists are typically sphingosine analogues, such as 2-substituted 2-amino-propane-1,3-diol or 2-amino-propanol derivatives. Examples of appropriate S1P receptor agonists are, for example:

Compounds as disclosed in EP 627406A1, e.g. a compound of formula I

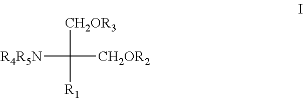

wherein
R₁ is a straight- or branched (C₁₂₋₂₂) carbon chain,
   which may have in the chain a bond or a hetero atom selected from a double bond, a triple bond, O, S, NR₆, wherein R₆ is H, alkyl, aralkyl, acyl or alkoxycarbonyl, and carbonyl; and/or
which may have as a substituent alkoxy, alkenyloxy, alkynyloxy, aralkyloxy, acyl, alkylamino, alkylthio, acylamino, alkoxycarbonyl, alkoxycarbonylamino, acyloxy, alkylcarbamoyl, nitro, halogen, amino, hydroxyimino, hydroxy or carboxy; or
R₁ is a phenylalkyl wherein alkyl is a straight- or branched (C₆₋₂₀)carbon chain; or
   a phenylalkyl wherein alkyl is a straight- or branched (C₁₋₃₀)carbon chain wherein said phenylalkyl is substituted by
      a straight- or branched (C₆₋₂₀)carbon chain optionally substituted by halogen,
      a straight- or branched (C₆₋₂₀)alkoxy chain optionally substituted by halogen,
      a straight- or branched (C₆₋₂₀)alkenyloxy;
   phenylalkoxy, halophenylalkoxy, phenylalkoxyalkyl, phenoxyalkoxy or phenoxyalkyl;
   cycloalkylalkyl substituted by a straight- or branched (C₆₋₂₀)alkyl chain;
   heteroarylalkyl substituted by a straight- or branched (C₆₋₂₀)alkyl chain;
   heterocyclic alkyl wherein said alkyl is a straight- or branched (C₆₋₂₀)carbon chain; or
   heterocyclic alkyl substituted by a straight- or branched (C₂₋₂₀)alkyl chain, and wherein the alkyl moiety may have in the carbon chain, a bond or a heteroatom selected from a double bond, a triple bond, O, S, sulfinyl, sulfonyl, or NR₆, wherein R₆ is as defined above;
and as a substituent alkoxy, alkenyloxy, alkynyloxy, aralkyloxy, acyl, alkylamino, alkylthio, acylamino, alkoxycarbonyl, alkoxycarbonylamino, acyloxy, alkylcarbamoyl, nitro, halogen, amino, hydroxy or carboxy, and
each of R₂, R₃, R₄ and R₅, independently, is H, C₁₋₄ alkyl or acyl
   or a pharmaceutically acceptable salt thereof;

Compounds as disclosed in EP 1002792A, e.g. a compound of formula II

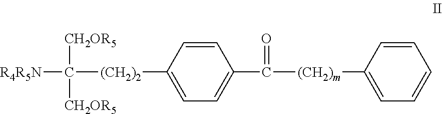

wherein
m is 1 to 9; and
each of R₂, R₃, R₄ and R₅, independently, is H, alkyl or acyl;
   or a pharmaceutically acceptable salt thereof;

Compounds as disclosed in EP 0778263 A1, e.g. a compound of formula III

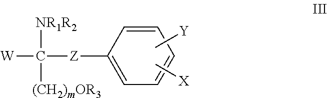

wherein
W is H; straight chain or branched (C₁₋₆)alkyl, (C₂₋₆)alkenyl or (C₂₋₆)alkynyl; unsubstituted or by OH substituted phenyl; R₄O(CH₂)ₙ; or straight chain or branched (C₁₋₆)alkyl substituted by 1 to 3 substituents selected from the group consisting of halogen, cycloalkyl, phenyl or phenyl substituted by OH;
X is H or unsubstituted or substituted straight chain alkyl having a number p of carbon atoms or unsubstituted or substituted straight chain alkoxy having a number (p−1) of carbon atoms, e.g. substituted by 1 to 3 substitutents selected from the group consisting of alkyl, OH, alkoxy, acyloxy, amino, alkylamino, acylamino, oxo, haloalkyl, halogen, unsubstituted phenyl or phenyl substituted by 1 to 3 substituents selected from the group consisting of alkyl, OH, alkoxy, acyl, acyloxy, amino, alkylamino, acylamino, haloalkyl and halogen;
Y is H, alkyl, OH, alkoxy, acyl, acyloxy, amino, alkylamino, acylamino, haloalkyl or halogen, Z is a single bond or a straight chain alkylene having a number or carbon atoms of q,
each of p and q, independently, is an integer of 1 to 20, with the proviso of 6≦p+q≦23,
m is 1, 2 or 3,
n is 2 or 3,
each of R₁, R₂, R₃ and R₄, independently, is H, alkyl or acyl,
   or a pharmaceutically acceptable salt thereof.

Compounds as disclosed in WO 02/18395, e.g. a compound of formula IVa or IVb

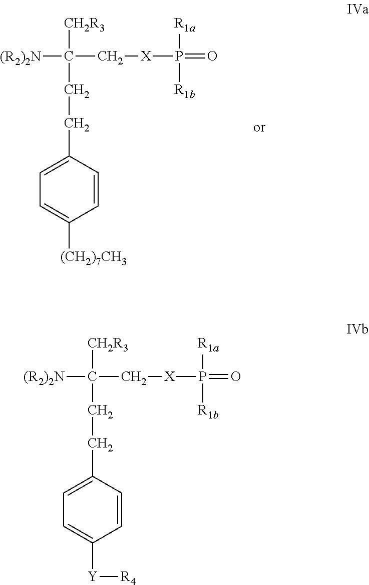

wherein
X is O, S, NR₁ or a group —(CH₂)ₙ—, which group is unsubstituted or substituted by
1 to 4 halogen;
n is 1 or 2,
R₁ is H or (C₁₋₄ alkyl, which alkyl is unsubstituted or substituted by halogen;
R₁ₐ is H, OH, (C₁₋₄)alkyl or O(C₁₋₄)alkyl wherein alkyl is unsubstituted or substituted by 1 to 3 halogen;
R_{1b} is H, OH or (C₁₋₄)alkyl, wherein alkyl is unsubstituted or substituted by halogen;
each R₂ is independently selected from H or (C₁₋₄)alkyl, which alkyl is unsubstituted or substituted by halogen;
R₃ in case of a compound of formula IVa is H, OH, halogen or O(C₁₋₄)alkyl wherein alkyl is unsubstituted or substituted by halogen;
R₃ in case of a compound of formula IVb is H, OH, halogen, (C₁₋₄)alkyl wherein alkyl is unsubstituted or substituted by hydroxy, or O(C₁₋₄)alkyl wherein alkyl is unsubstituted or substituted by halogen,
Y is —CH₂—, —C(O)—, —CH(OH)—, —C(═NOH)—, O or S,
R₄ is (C₄₋₁₄)alkyl or (C₄₋₁₄)alkenyl;
   or a pharmaceutically acceptable salt or hydrate thereof;

Compounds as disclosed in WO 02/06268 or JP-14316985, e.g. a compound of formula VII

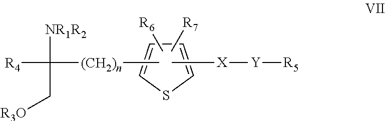

wherein
each of R₁ and R₂, independently, is H or an amino-protecting group;
R₃ is hydrogen or a hydroxy-protecting group;
R₄ is (C₁₋₆)alkyl;
n is an integer of 1-6;
X is ethylene, vinylene, ethynylene, a group having a formula -D-CH₂— (wherein, D is carbonyl, a group having a formula —CH(OH)—, O, S or N; aryl or aryl substituted by three members selected from group a as defined hereinafter;
Y is single bond, C₁₋₁₀alkylene, C₁₋₁₀alkylene which is substituted by one to three substituents selected from groups a and b, C₁₋₁₀alkylene having 0 or S in the middle or end of the carbon chain, or C₁₋₁₀alkylene having 0 or S in the middle or end of the carbon chain which is substituted by one to three substituents selected from groups a and b;
R₅ is hydrogen, cycloalkyl, aryl, heterocycle, cycloalkyl substituted by one to three members selected from groups a and b, aryl substituted by one to three members selected from groups a and b, or heterocycle substituted by one to three members selected from groups a and b; and
each of R₆ and R₇, independently, is H or a substituent selected from group a;
<group a> is halogen, lower alkyl, halogeno lower alkyl, lower alkoxy, lower alkylthio, carboxyl, lower alkoxycarbonyl, hydroxy, lower aliphatic acyl, amino, mono-lower alkylamino, di-lower alkylamino, lower aliphatic acylamino, cyano and nitro;
<group b> is cycloalkyl, aryl, heterocycle, each being optionally substituted by up to three substituents selected from group a;
with the proviso that when R₅ is hydrogen, Y is either a single bond or linear C₁₋₁₀ alkylene,
e.g. (2R)-2-amino-4-[3-(4-cyclohexyloxybutyl)benzo[b]thien-6-yl]-2-methylbutan-1-ol,
   or a pharmacologically acceptable salt or ester thereof.

When in the compounds of formula I the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy. Acyl may be a residue R—CO—, wherein R is C₁₋₆alkyl, C₃₋₄cycloalkyl, phenyl or phenyl-C₁₋₄alkyl.

Preferred compounds of formula I are those wherein R₁ is a straight or branched, preferably straight, chain alkyl having 13-20 carbon atoms, optionally substituted by nitro, halogen, amino, hydroxy or carboxy, and, more preferably those wherein R₁ is phenylalkyl substituted by a straight or branched C₆₋₁₄alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

A preferred compound of formula I is 2-amino-2-tetradecyl-1,3-propanediol. A particularly preferred S1P receptor agonist of formula I is 2-amino-2-[2-(4-octylphenylethyl)]propane-1,3-diol in free form or in a pharmaceutically acceptable salt form (referred to hereinafter as Compound A), e.g. the hydrochloride, i.e. FTY720, as shown:

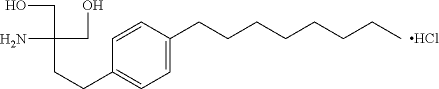 [SMILES: CCCCCCCCc1ccc(CCC(N)(CO)CO)cc1 ; Cl]

A preferred compound of formula II is one wherein each of R₂ to R₅ is H and m is 4, i.e. 2-amino-2-{2-[4-(1-oxo-5-phenylpentyl)phenyl]ethyl}propane-1,3-diol (referred to hereinafter as Compound B), in free form or in a pharmaceutically acceptable salt form, e.g. the hydrochloride.

A preferred compound of formula IVa is the Compound A-phosphate (R₂ is H, R₃ is OH, X is O, R₁ₐ and R_{1b} are OH). A preferred compound of formula V is Compound B-phosphate (R₁ is CH₂OH, R₃ is H, X is O, m is 1, R₂ is phosphate and R is 2-[4-(1-oxo-5-phenylpentyl)phenyl]ethyl).

When the compounds of formulae I to VII have one or more asymmetric centers in the molecule, the various optical isomers, as well as racemates, diastereoisomers and mixtures thereof are embraced.

Examples of pharmaceutically acceptable salts of the compounds of formulae I to VII include salts with inorganic acids, such as hydrochloride, hydrobromide and sulfate, salts with organic acids, such as acetate, fumarate, maleate, benzoate, citrate, maleate, methanesulfonate and benzenesulfonate salts, or, when appropriate, salts with metals, such as sodium, potassium, calcium and aluminium, salts with amines, such as triethylamine and salts with dibasic amino acids, such as lysine. The compounds and salts of the present invention encompass hydrate and solvate forms.

The composition of the present invention may comprise one or more salts and/or free acid of the S1P modulator.

The composition of the invention preferably contains 0.01 to 20% by weight of S1P receptor modulator, more preferably 0.1 to 10%, e.g. 0.5 to 5% by weight, based on the total weight of the composition.

Where the pharmaceutical capsule is in unit dosage form, each unit dosage may suitably contain 0.5 to 10 mg of the S1P receptor modulator.

Use

Pharmaceutical compositions of the present invention are useful, either alone or in combination with other active agents, for the treatment and prevention of conditions e.g. as disclosed in U.S. Pat. No. 5,604,229, WO 97/24112, WO 01/01978, U.S. Pat. No. 6,004,565, U.S. Pat. No. 6,274,629 and JP-14316985, the contents of which are incorporated herein by reference.

The compositions described herein may promote the absorption and distribution of the S1P modulator through the blood brain barrier and into the brain.

In particular, the pharmaceutical compositions are useful for:

a) treatment and prevention of organ or tissue transplant rejection, for example for the treatment of the recipients of heart, lung, combined heart-lung, liver, kidney, pancreatic, skin or corneal transplants, and the prevention of graft-versus-host disease, such as sometimes occurs following bone marrow transplantation; particularly in the treatment of acute or chronic allo- and xenograft rejection or in the transplantation of insulin producing cells, e.g. pancreatic islet cells;
b) treatment and prevention of autoimmune disease or of inflammatory conditions, e.g. multiple sclerosis, arthritis (for example rheumatoid arthritis), inflammatory bowel disease, hepatitis, etc.;
c) treatment and prevention of viral myocarditis and viral diseases caused by viral mycocarditis, including hepatitis and AIDS.

The invention is, in one embodiment, related to the treatment of inflammatory conditions. In one example, the invention is related to compositions for the control and/or suppression of mast cell activation and secretion for the relief of inflammatory conditions, e.g. in the brain as in multiple sclerosis.

There is also provided a method of protecting multiple sclerosis subjects against neurodegerative brain inflammation, comprising the administration to said subjects a composition as described herein, for example a composition comprising an S1P agonist or other modulator.

Compositions of the present invention and any concentrate for dilution and pharmaceutical solution made therefrom, may be administered in an amount which is therapeutically effective against a disease or condition which can be treated by administration of the S1P receptor modulator.

The exact amount of S1P receptor modulator or pharmaceutically acceptable salt thereof to administer can vary widely. The dose may depend on the particular compound, route of administration, the rate of administration, the strength of the particular concentrate or pharmaceutical solution employed, the nature of the disease or condition being treated, and the sex, age and body weight of the patient. The dose may also depend on the existence, nature and extent of any adverse side-effects that may accompany the administration of the concentrate or pharmaceutical formulation. Typically, a dose of 0.5 to 5 mg of S1P receptor modulator, e.g. Compound A, are administered to children.

The composition of the present invention and any concentrate for dilution and respective pharmaceutical solution may be used in combination with other immunosuppressant(s), steroid(s) such as prednisolone, methylprednisolone, dexamethasone, hydrocortisone and the like, or nonsteroidal anti-inflammatory agent. The administration of a combination of active agents may be simultaneous or consecutive, with either one of the active agents being administered first. The dosage of the active agents of a combination treatment may depend on effectiveness and site of action of each active agent, as well as synergistic effects between the agents used for combination therapy.

The invention will now be described with reference to the following specific Examples.

### EXAMPLE 1

Micronized Compound 2-amino-2-[2-(4-octylphenyl)ethyl]propane-1,3-diol, hydrochloride salt (FTY720), is screened mixed with the microcrystalline cellulose agent, e.g. Avicel PH 102. The mixture is then milled in a Frewitt MGI device (Key International Inc. USA) using a 30 mesh screen. Magnesium stearate is screened using a 20 mesh screen and blended with the FTY720/cellulosemixture. Crosscarmellose is the blended to produce a product composition.

An example for a 6 mm round, 80 mg tablet core obtained by direct compression is shown below:

| Ingredient | mg/dose |
|---|---|
| FTY720 HCl | 1.40 |
| Microcrystalline cellulose, e.g. Avicel PH 102 | 73.80 |
| Magnesium stearate | 0.80 |
| Crosscarmellose | 4.00 |

As an alternative, a core tablet composition may be compacted on a tablet press using a 7 mm die to form 120 mg tablets, an example of which may be:

| Ingredient | mg/dose |
|---|---|
| FTY720 HCl | 1.40 |
| Mannitol M200 | 116.20 |
| Magnesium stearate | 2.40 |

1 mg of FTY720 in free form is equivalent to 1.12 mg of FTY720 HCl salt.

### EXAMPLE 2

In a further example, the process of Example 1 is repeated except that the magnesium stearate is replaced by Cutina® (hydrogenated castor oil).

### EXAMPLE 3

In a further Example, the tablets are prepared as described in Examples 1 and 2, except that FTY720 is replaced in each case by 2-amino-2-{2-[4-(1-oxo-5-phenylpentyl)phenyl]ethyl}propane-1,3-diol hydrochloride.

### EXAMPLES 4 TO 7

Tablets containing the following ingredients (in mg) are produced:

| | Example 4 | Example 5 | Example 6 | Example 7 |
|---|---|---|---|---|
| FTY720 | 1 | 1 | 1 | 1 |
| D-mannitol | 62.3 | 62.3 | 62.0 | 62.0 |
| Xylitol* | 26.7(5.4) | 26.7(5.4) | 26.6 | 26.6 |
| Methylcellulose | — | — | 0.4 | 0.4 |
| Microcrystalline | 24.0 | — | 24.0 | — |
| cellulose | | | | |
| Low-substituted | — | 24.0 | — | 24.0 |
| Hydroxypropyl- | | | | |
| cellulose | | | | |
| Hydrogenated oil | 6.0 | 6.0 | 6.0 | 6.0 |
| Total | 120.0 | 120.0 | 120.0 | 120.0 |

*The amount of xylitol indicated in brackets was used as a binder.

FTY720, D-mannitol and xylitol are placed in a fluid-bed granulator (MP-01 model, Powrex), mixed for five minutes, and granulated under spray of binder solution, followed by drying till the exhaust temperature reaches 40° C. The granulation conditions are as shown below. Dried powder is passed through a 24-mesh sieve, added to the specified amount of filler and lubricant, and mixed in a mixer (Tubular Mixer, WAB) for three minutes to make the powder for compression.

The resulting powder is compressed by a tabletting machine (Cleanpress correct 12 HUK, Kikushui Seisakusho) with a punch of 7 mm i.d. x 7.5 mm R at a compression force of 9800 N.

Granulation conditions:

| Item | Setting | |
|---|---|---|
| Charge-in amount | 1170 | g |
| Volume of intake-air | 50 | m³/min |
| Temperature of intake-air | 75° | C. |
| Flow rate of spray solution | 15 | mL/min |
| Spray air pressure | 15 | N/cm² |
| Spray air volume | 30 | L/min |
| Volume of binder solution | 351 | mL |

### EXAMPLE 8

An example powder coating composition:

The components are premixed under high shear, then wet granulated by mixing under high shear with water. The granulated mixture is dried in fluid bed drier to reduce the moisture content to below 3% by weight. The dried granules are milled and micronised to a powder.

| Ingredient | Composition (% w/w) |
|---|---|
| Ammonio-methacrylate co-polymer, e.g. | 46.5 |
| Eudragit RS | |
| hydroxy propyl cellulose, e.g. Klucel | 28.0 |
| titanium dioxide | 15.0 |
| aluminium lake | 5.0 |
| polyethylene glycol 6000 | 5.0 |
| colloidal silicon dioxide, e.g. Aerosil 200 | 0.5 |

### EXAMPLE 9

An example powder coating composition:

| Ingredient | Composition (% w/w) |
|---|---|
| Ammonio-methacrylate co-polymer, e.g. | 39.75 |
| Eudragit RS | |
| hydroxy propyl cellulose, e.g. Klucel | 39.75 |
| titanium dioxide | 15.0 |
| aluminium lake | 5.0 |
| colloidal silicon dioxide, e.g. Aerosil | 0.5 |

### EXAMPLE 10

An example liquid coating composition (aqueous dispersion):

At the fusing or drying stations, energy is imparted to the core surfaces to fuse the powder or dry the liquid and provide a uniform coating on the exposed surfaces of the core. The energy is provided by focused radiation preferably in the infra-red region; the energy power requirement will be determined largely by the coating material. After fusing or drying, the coating is set by cooling, using an air blower.

| Ingredient | Composition (% w/w) |
|---|---|
| hydroxypropylmethylcellulose | 70 |
| glycerol | 7 |
| iron oxide yellow | 23 |

### EXAMPLE 10

An example for a 7 mm round, 127 mg tablet for fast disintegration according to the present invention:

| Ingredient | mg/dose |
|---|---|
| FTY720 HCl | 0.56 |
| Directly compressible mannitol, e.g. Parteck | 82.54 |
| M200 | |
| Calcium silicate | 36.00 |
| Magnesium stearate | 0.90 |
| Crospovidone | 7.00 |

The tablet may be manufactured by known methods. For example, the tablet may be manufactured by blending of all ingredients and further compressing to tablets and/or granulation and/or micronisation and further compressing of the granules to tablets.

### EXAMPLE 11

A rapid disintegrating formulation is prepared, which comprises gelatin (3%), mannitol as structure forming agent (1-5%), sweeteners, flavoring agents.

Gelatin and mannitol are added to the water and heated to 40° C. to dissolve. The gelatin/mannitol solution is cooled to 23° C. and mixed with the active ingredient, e.g. an S1P agonist or other modulator. The total solid content is less than 50%. The suspension is first cooled to 15° C. to prevent sedimentation of the suspension before the start of the lyophilization (coated or uncoated).

### EXAMPLE 12

As Example 11, except where the mannitol is replaced with sorbitol.

### EXAMPLE 13

Micronized compound 2-amino-2-[2-(4-octylphenyl)ethyl]propane-1,3-diol, hydrochloride salt (FTY720), is screened mixed with the microcrystalline cellulose agent, e.g. Avicel PH 102. The mixture is then milled in a Frewitt MGI device (Key International Inc. USA) using a 30 mesh screen. Magnesium stearate is screened using a 20 mesh screen and blended with the FTY720/cellulosemixture. Crosscarmellose is the blended to produce a product composition.

An example for a 6 mm round, 80 mg tablet core obtained by direct compression is shown below:

| Ingredient | mg/dose |
|---|---|
| FTY720 HCl | 1.40 |
| Microcrystalline cellulose, e.g. Avicel PH 102 | 73.80 |
| Magnesium stearate | 0.80 |
| Crosscarmellose | 4.00 |

### EXAMPLE 14

Micronized Compound A, e.g. 2-amino-2-[2-(4-octylphenyl)ethyl]propane-1,3-diol, hydrochloride salt (FTY720), is screened and 116.7 g of the screened compound is mixed with 9683.3 g of a microcrystalline cellulose agent. The mixture is then milled in a Frewitt MGI device (Key International Inc. USA) using a 30 mesh screen. Magnesium stearate is screened using a 20 mesh screen and 200 g of the screened compound blended with the FTY720 mixture to produce a product composition.

The product composition is then compacted on a tablet press using a 7 mm die to form 120 mg tablets, each containing:

| | |
|---|---|
| Compound A, e.g. FTY720 * | 1.4 mg |
| Microcrystalline cellulose, e.g. Avicel PH 102 | 116.2 mg |
| Magnesium stearate | 2.4 mg |
| Total | 120 mg |

* 1 mg of Compound A in free form is equivalent to 1.12 mg of FTY720.

### EXAMPLE 15

In a further example, the process of Example 14 is repeated except that the magnesium stearate is replaced by Cutina® (hydrogenated castor oil).

### EXAMPLE 16

Compound A, e.g. FTY720, and microcrystalline cellulose, e.g. Avicel PH 102 are each screened separately using an 18 mesh screen. 1.9 g screened FTY720 is mixed with 40 g screened microcrystalline cellulose agent for 120 revolutions in a blender at 32 rpm. The FTY720 mixture is then screened through a 35 mesh screen.

The screened FTY720 mixture is added to a granulator along with a further 340.1 g Microcrystalline cellulose, e.g. Avicel PH 102 and 12 g hydroxypropylcellulose. The mixture is mixed for 3 minutes. Water is then added at a rate of 100 ml/minute and the mixture granulated for 2 minutes. The granulation is transferred into a tray dryer and dried at 50° C. for 150 minutes.

The mixture is then milled in a Frewitt MGI device using a 35 mesh screen. Magnesium stearate is screened and 6 g of the screened compound is blended for 90 revolutions at 32 rpm with the FTY720 mixture to produce a product composition showing a substantially uniform distribution of the S1P receptor agonist throughout the microcrystalline cellulose, e.g. Avicel PH 102 in the blend.

The product composition is then filled into size 3 hard gelatin shells on an H & K 400 encapsulation device. 120 mg of the product composition is added to each capsule. Therefore each capsule contains:

| | |
|---|---|
| FTY720 * | 0.56 mg |
| Microcrystalline cellulose | 114.04 mg |
| Hydroxypropylcellulose | 3.6 mg |
| Magnesium stearate | 1.8 mg |
| Total | 120 mg |

### EXAMPLE 17

In a further example, the process of Example 16 is repeated except that the magnesium stearate is replaced by Cutina® (hydrogenated castor oil).

### EXAMPLE 18

In a further example, the process of Example 16 is repeated except that the hydroxypropyl cellulose is replaced by hydroxypropylmethyl cellulose.

### EXAMPLE 19

Micronized Compound A, e.g. FTY720, is screened using a 425 μm (40 mesh) screen. 58.35 g of the screened compound is mixed with 4841.65 g microcrystalline cellulose, e.g. Avicel PH 102 in a 25L Bohle bin blender for 240 blending revolutions. The mixture is then milled in a Frewitt MGI device using a 425 μm mesh screen, and the milled mixture is blended once more. Magnesium stearate is screened and 100 g of the screened compound is blended with the FTY720 mixture to produce a product composition showing a substantially uniform distribution of the S1P receptor agonist throughout the blend.

The product composition is then filled into size 3 hard gelatin shells on an H & K 400 encapsulation device. 120 mg of the product composition is added to each capsule. Therefore each capsule contains:

| | |
|---|---|
| FTY720 * | 1.4 mg |
| Microcrystalline cellulose | 116.2 mg |
| Magnesium stearate | 2.4 mg |
| Total | 120 mg |

### EXAMPLES 20 AND 21

In further examples, capsules are prepared as described in Example 19, except that each capsule contains each component in the following amounts:

| | |
|---|---|
| Example 20 | Example 21 |

| | | | | |
|---|---|---|---|---|
| FTY720 * | 2.8 | mg | 5.6 | mg |
| Microcrystalline cellulose | 114.8 | mg | 112 | mg |
| Magnesium stearate | 2.4 | mg | 2.4 | mg |
| Total | 120 | mg | 120 | mg |

### EXAMPLES 22 TO 24

In further examples, capsules are prepared as described in Examples 19 to 21, except that the magnesium stearate is replaced in each case by Cutina® (hydrogenated castor oil).

### EXAMPLES 25 TO 35

In further examples, capsules or tablets are prepared as described in Examples 13 to 23, except that FTY720 is replaced in each case by 2-amino-2-(2-[4-(1-oxo-5-phenylpentyl)phenyl)ethyl]propane-1,3-diol hydrochloride.

### EXAMPLES 36 TO 38

Pharmaceutical compositions containing the following ingredients are produced:

| | Example 36 | | Example 37 | | Example 38 | |
|---|---|---|---|---|---|---|
| FTY720 | 5 | g | 10 | g | 100 | g |
| Microcrystalline cellulose | 991 | g | 986 | g | 897 | g |
| Methylcellulose SM-25 | 4 | g | 4 | g | 3 | g |
| Total | 1000 | g | 1000 | g | 1000 | g |

The FTY720 and a proportion of the microcrystalline cellulose, e.g. Avicel PH 102 equal to twice the weight of the FTY720 are mixed in a Microspeed Mixer MS-5 type (Palmer, USA) for 2 minutes at 1200 rpm. The remaining microcrystalline cellulose is added to the mixture and mixed for another 2 minutes. 80 or 60 milliliters of 5% methylcellulose SM-25 solution is supplied from a hopper and granulated under the same conditions. The mixture is extruded through a screen with 0.4 mm apertures using an extruder RG-5 type. The extruded material is dried at 65° C. by a fluidized-bed granulator STREA I Type (Patheon, Canada) and then sieved through a 24 mesh sieve. Fine particles which pass through a 60 mesh sieve are removed. The obtained fine granules are filled into capsules by a Zuma capsule-filling machine (100 mg per capsule).

### EXAMPLE 39

An example of a tablet formulation comprising 1.25 mg FTY720 obtainable by wet granulation.

Composition for wet granulation:

| Ingredient | mg/tablet | % |
|---|---|---|
| FTY HCl | 1.49 | 1.49 |
| HPMC 3cps | 3.00 | 3.00 |
| Water granulation liquid | q.s | q.s |
| Mannitol | 46.25 | 46.25 |
| Avicel PH 101 | 46.25 | 46.25 |
| Aerosil 200 | 3.01 | 3.01 |
| Croscarmellose | 5.00 | 5.00 |
| Magnesium stearate | 1.00 | 1.00 |
| Total | 100.00 | 100.00 |

Microcrystalline cellulose was wet granulated with an aqueous solution of FTY720 and HPMC. After drying, the mixture was sieved and blended with mannitol, silicon dioxide, croscarmellose and magnesium stearate, and compressed into 6 mm round tablets of 100 mg.

This formulation can alternatively be manufactured without sugar alcohols such as mannitol, using microcrystalline cellulose instead:

| Ingredient | mg/tablet | % |
|---|---|---|
| FTY HCl | 1.49 | 1.49 |
| HPMC 3cps | 3.00 | 3.00 |
| Water granulation liquid | q.s | q.s |
| Avicel PH 101 | 92.50 | 92.50 |
| Aerosil 200 | 3.01 | 3.01 |
| Croscarmellose | 5.00 | 5.00 |
| Magnesium stearate | 1.00 | 1.00 |
| Total | 100.00 | 100.00 |

### EXAMPLE 40

An example of coating composition comprising FTY720.

Composition for coating of pellets, minitablets and small tablets

| Ingredient | mg/tablet | % |
|---|---|---|
| HPMC 3cps | 1.62 | 11.60 |
| FTY HCl | 0.04 | 0.25 |
| Butylhydroxytoluol | 0.01 | 0.05 |
| Triethylcitrate | 0.07 | 0.50 |
| Acetone | 6.12 | 43.81 |
| Ethanol | 6.12 | 43.81 |
| Total Dry | 1.74 | 12.39 |
| Total | 100.00 | 100.00 |

The polymer HPMC can also be replaced by, for example, HPC or other comparable polymers. The FTY720 coat can be applied to active or placebo pellets, minitablets or small tablets separated by, for example, a protection coat (e.g. HPMC) and/or covered with an overcoat (e.g. HPMC). This dosage form can be filled into capsules (e.g. HPMC or HGC) or stickpacks and hence is flexible in the sense that different dosage strengths or combination products may be formulated.

1. A fast disintegrating solid pharmaceutical composition comprising:
(a) an S1P receptor modulator
(b) an alkaline earth metal silicate
(c) a disintegration agent
wherein the ratio of the silicate:disintegration agent is from 2:1 to 10:1

2. A composition according to claim 1, where the ratio is 3:1 to 7:1.

3. A composition according to claim 2, where the ratio is 5:1.

4. A composition according to claim 1, wherein the disintegration agent is selected from crospovidone and croscarmellose.

5. A composition according to claim 1, wherein the disintegration time is less than 60 seconds.

6. A rapid disintegrating pharmaceutical composition comprising a freeze dried dosage form of an S1P receptor modulator.

7. A composition according to claim 6, additionally comprising one or more of gelatin, mannitol, sorbitol, dextrose, sucrose, lactose, maltose, maltodextrins, corn syrup solids, trehalose, polyvinyl pyrrolidone, polyelectrolyte gel A chondroitin sulfate, cellulose, starch derivatives, Pullulan, glycine, docusate Na, PVC, HPC-SL, mannitol & glycerol, gum xanthan/carragean/acacia/guar/tragacanth, mannitol, polysorbate 60, sodium dodecylsulfate, fatty acids, bile salts, sodium methylhydroxybenzoate, sodium propylhydroxybenzoate, viscosity enhancers, flavoring agents, sweeteners.

8. A composition according to claim 6, wherein the disintegration time is less than 10 seconds.

9. A solid pharmaceutical composition suitable for oral administration, comprising:
(a) an S1P receptor modulator; and
(b) a microcrystalline cellulose
in the absence of a sugar alcohol.

10. A composition according to claim 9, comprising 90 to 99.5% by weight of the microcrystalline cellulose.

11. A composition according to claim 9, wherein the microcrystalline cellulose comprises Avicel®.

12. A pharmaceutical composition which comprises a coating comprising an S1P receptor modulator.

13. A composition according to claim 12, wherein the composition comprises a core coated with said coating.

14. A composition according to claim 13, wherein the core comprises a granule, pellet, tablet or minitablet.

15. A composition according to claim 13, wherein the core comprises an S1P receptor modulator.

16. A composition according to claim 12, wherein the coating further comprises a polymer.

17. A composition according to claim 16, wherein the polymer comprises a cellulose.

18. A composition according to claim 17, wherein the polymer comprises hydroxypropyl methylcellulose, hydroxypropyl cellulose or methyl cellulose.

19. A composition according to claim 12, which comprises one or both of ethanol and acetone.

20. A composition according to claim 12, wherein the coating comprises:

| Ingredient | % |
|---|---|
| Hydroxypropylmethylcellulose (HPMC) | 11.60 |
| S1P receptor modulator, e.g. FTY HCl | 0.25 |
| Butylhydroxytoluol | 0.05 |
| Triethylcitrate | 0.50 |
| Acetone | 43.81 |
| Ethanol | 43.81 |

21. A composition according to claim 12, wherein the composition comprises one or more additional coatings.

22. A process for producing a pharmaceutical composition, comprising
(a) mixing a freeze dried dosage form of an S1P receptor modulator with a structure forming agent;
(b) producing an aqueous suspension, wherein the aqueous suspension contains less than 50% solid; and
(c) optionally further conducting a lyophillisation step.
